(19) United States
(12) Patent Application Publication
(10) Pub. No.: US 2026/0294456 A1
(43) Pub. Date: Oct. 1, 2026

(54) SURGICAL CLAMP FOR SIMULTANEOUS GRASPING AND SPATIAL ACCESS TO CLAMPED TISSUE

(71) Applicant: Teo Soleymani, La Canada Flintridge, CA (US)

(72) Inventor: Teo Soleymani, La Canada Flintridge, CA (US)

(21) Appl. No.: 19/306,355

(22) Filed: Aug. 21, 2025

Related U.S. Application Data
(60) Provisional application No. 63/696,057, filed on Sep. 18, 2024.

(51) Int. Cl. A61B 17/28; A61B 17/00

(52) U.S. Cl. CPC A61B 17/282; A61B 17/2812; A61B 2017/00858; A61B 2017/2808

(57) ABSTRACT

A surgical clamp comprising a handle portion and a partial loop grasping head portion oriented in a non-planar configuration relative to the handle portion. The partial loop grasping head portion features a round or ovoid shape with distal jaw portions configured to provide access for suturing or stapling tissue, and an inner clamping surface. The clamp's design allows for both proximal and lateral clamping of anatomical structures while maintaining lateral access aperture, enhancing the precision and effectiveness of ligation procedures. The partial loop grasping head opens laterally, either to the left or right, providing improved access in confined or delicate areas during surgical procedures.

### PRIORITY

This application claims the benefit of U.S. Provisional Application No. 63/696,057, filed Sep. 18, 2024, the content of which is herein incorporated by reference in its entirety.

### TECHNICAL FIELD

The technical field of this invention relates to surgical instruments, specifically to clamping devices used in surgical procedures for ligation of anatomical structures such as arteries, veins, and nerves.

### BACKGROUND

Surgical procedures require precise control and manipulation of anatomical structures such as arteries, veins, and nerves. Traditional surgical clamps and hemostats have been widely used for these purposes, but present limitations in certain scenarios, particularly in confined or delicate areas.

Some conventional clamping devices may provide a full circular grip around the intended structure. While effective in many situations, this design can be obstructive or less effective in confined spaces or when dealing with delicate tissues. Moreover, these traditional designs often make it challenging to perform ligation procedures, as the instrument itself may interfere with the placement of sutures or staples.

The need for more specialized surgical instruments has become increasingly apparent, especially in procedures requiring lateral access or precise directional control of clamping pressure. Furthermore, the success rate of ligation procedures is dependent on the clear exposure and accessibility of the distal end of the structure being ligated. Traditional clamping devices often fall short in this aspect, as they may obstruct the surgeon's view or access to the critical ligation point.

### BRIEF DESCRIPTION OF THE SEVERAL VIEWS OF THE DRAWINGS

The drawings illustrate generally, by way of example, but not by way of limitation, various embodiments discussed herein.

FIGS. 1A-1B are diagrams depicting a surgical clamp, according to certain examples.

FIGS. 2A-2B are diagrams depicting a surgical clamp held by a user, according to certain examples.

FIGS. 3A-3C are diagrams depicting a partial loop grasping head of a surgical clamp, according to certain examples.

FIGS. 4A-4D are diagrams depicting an inner surface of a partial loop grasping head of a surgical clamp, according to certain examples.

FIGS. 5A-5B are diagrams depicting a surgical clamp, according to certain examples.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

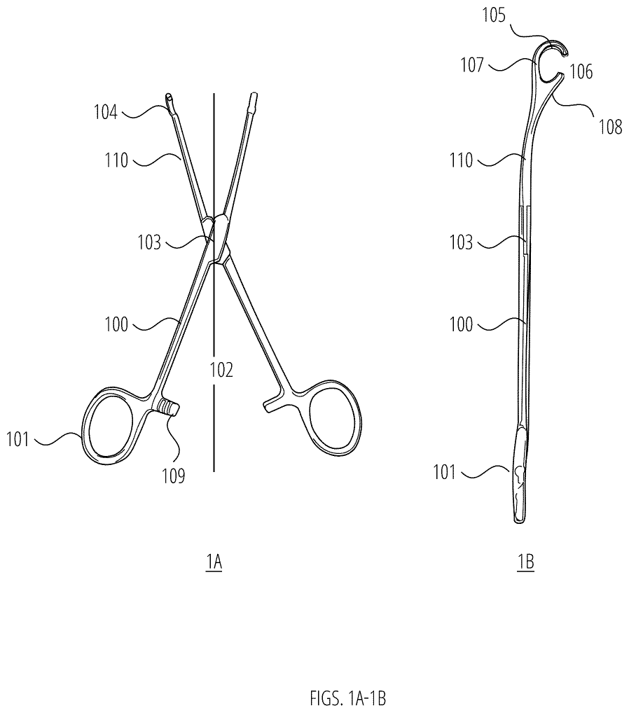

FIG. 1A and 1B provide diagrams depicting a surgical clamp, according to certain examples. As seen in FIGS. 1A and 1B, the surgical clamp may comprise proximal first and second handle portions 101 connected to first and second shafts 100, which conjoin at a hinge 103.

FIG. 1A provides a front view of the exemplary surgical clamp showing the proximal first and second handle portions 101 connected to first and second shafts 100, which conjoin at a hinge 103. The first and second handle portions 101 are designed for comfortable gripping and manipulation of the instrument during surgical procedures. The first and second shafts 100 extend distally from the handle portions and meet at the hinge 103, which enables the pivoting movement necessary for the clamping action. The hinge 103 is positioned at the medial portion of the shafts 100 and is configured to interleave the first and second shafts. The distal ends of the first and second shafts 100 terminate in the first and second jaw portions 104, which meet along a grasping plane 102. The first and second jaw portions 104 further comprise first and second partial loop grasping head portions 105 that are defined by a lateral access aperture 106, perimeter 107, and one or more atraumatic edges 108. The partial loop grasping head portions 105 are designed with a C-shaped configuration that creates the grasping plane 102 when the jaws are closed. The first and second handle portions 101 also incorporate a ratchet mechanism 109 that provides incremental locking positions to maintain consistent clamping pressure without requiring continuous manual force from the surgeon.

FIG. 1B provides a side perspective view of the same surgical clamp, particularly illustrating the laterally curved distal shaft portions 110 and the lateral access aperture 106 of the partial loop grasping head portions 105. From this perspective, the non-planar configuration of the partial loop grasping head portions 105 relative to the first and second handle portions 101 is clearly visible, with the angular displacement between the tip and the longitudinal axis of the handle portions ranging from 0 to 90 degrees. The distal portions of the first and second shafts 100 include laterally curved distal shaft portions 110 that directly connect to first and second jaw portions 104. The first and second jaw portions 104 meet along the grasping plane 102 and further comprise first and second partial loop grasping head portions 105. The lateral access aperture 106 is prominently displayed in this view, showing how it facilitates unimpeded access to the clamped anatomical structure for suturing or stapling procedures. Also visible is the ratchet 109 connecting the first and second handle portions 101, which provides constant directional clamping pressure.

In some examples, the first and second partial loop grasping head portions 105 are positioned entirely on one lateral aspect of the laterally curved distal shaft portions 110. The distal shaft portions 110 connect to the proximal end or side of the C-shaped perimeter 107, resulting in an asymmetrical configuration.

In some examples, the first and second partial loop grasping head portions 105 may extend distally from the distal shaft portions 110, forming a unilateral clamping surface. This arrangement places the entire partial loop grasping head portion 105, including both the enclosed perimeter 107 and the open lateral access aperture 106, on the same side of the distal shaft portion 110.

The invention provides proximal and lateral clamping while leaving a lateral access aperture 106, which increases the success rate of ligation procedures. The distal tip of the partial loop grasping head portion 105 is freely exposed within the center lateral access aperture 106 without interference or obstruction from the perimeter 107.

In some examples, the first and second handle portions 101 comprise a ratchet 109, which provides constant directional clamping pressure.

In some examples, the ratchet mechanism exerts between 0.1-0.5 N/mm² (10-50 kPa), which is suitable for handling fragile tissues such as blood vessels, nerves, or thin membranes.

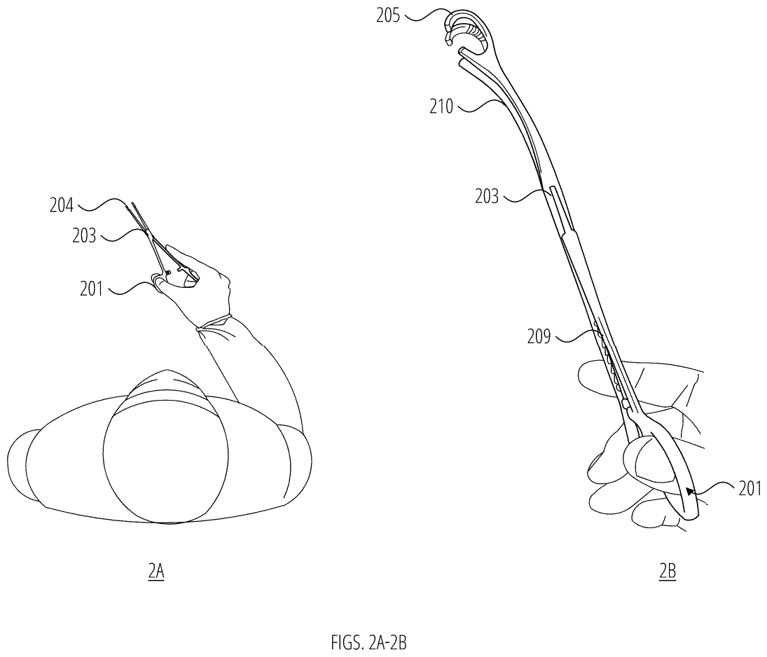

FIGS. 2A-2B provide diagrams depicting an exemplary surgical clamp held by a user, wherein the first and second handle portions 201 of the tool are circular, oval, or shaped in any manner that accommodates the size and shape of the user's fingers.

FIG. 2A provides a front view of the exemplary surgical clamp showing the first and second handle portions, while FIG. 2B provides a side perspective view that illustrates the laterally curved distal shaft portions and their connection to the first and second partial loop grasping head portions. According to certain examples, the first and second handle portions 201 are connected to laterally curved distal shaft portions 210, which in turn attach to the base of the first and second jaw portions 204 comprising first and second partial loop grasping head portions 205. The laterally curved distal shaft portions 210 are affixed to a nadir of the first and second jaw portions 204, effectively bisecting the tip into two distinct regions. A ratchet 209 optionally connects the first and second handle portions 201 when a user desires prolonged clamping capability.

In some examples, the laterally curved distal shaft portions 210 may be angled between 0 and 45 degrees relative to the longitudinal axis of the first and second handle portions 201 to provide improved clearance for the clamp during surgical procedures. On the lateral aspect of the laterally curved distal shaft portions 210, the enclosed segment of the first and second partial loop grasping head portions 205 extend superiorly, providing a robust clamping surface.

In some examples, the laterally curved first and second distal shaft portions 210 extend from the first and second handle portions 201 to the first and second jaw portions 204 and may be customizable in length and shape to meet specific surgical needs. For example, the length of the laterally curved distal shaft portions 210 can be configured according to surgical specialty, with options for both longer and shorter lengths depending on the procedural requirements.

In some examples, the shape of the distal shaft portions 210 can be adapted based on surgical location requirements and mechanical constraints of the tissue being clamped. The distal shaft portions 210 can be configured as either linear or curved, allowing surgeons to optimize access and manipulation in confined surgical spaces. This customization ensures that the invention can be tailored to meet the specific demands of various surgical specialties and individual surgeon preferences.

In some examples, the hinge 203 is configured to accept pivot, box, or spring mechanisms for interleaving of the first and second shafts. The hinge 203 is selected depending on the mechanical constraints of the tissue-type being clamped.

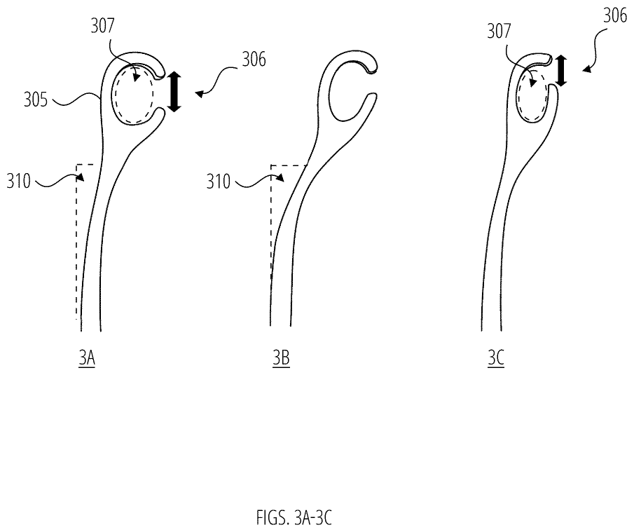

FIGS. 3A-3C are diagrams depicting a partial loop grasping head of a surgical clamp, according to certain examples.

FIG. 3A shows a side view of the surgical clamp illustrating the laterally curved distal shaft portions 310. The partial loop grasping head 305 is connected to the laterally curved distal shaft portion 310 and is defined by the lengths of both the lateral access aperture 306 and the perimeter 307, with a smooth inner surface configuration 311 of the partial loop grasping head portions.

FIG. 3B provides a view demonstrating the partial loop grasping head portions 305 designed to open laterally, either to the left or right side relative to the handle. This figure shows the same structural components of the surgical clamp depicted in FIG. 3A, but in a different exemplary configuration, wherein the lateral distal shaft portion 310 is curved at a larger angle in comparison to FIG. 3A.

FIG. 3C illustrates the lateral access aperture 306 and perimeter 307 configuration of the partial loop grasping head portions, showing how these features facilitate unimpeded access to the clamped anatomical structure. This figure shows another exemplary configuration of the surgical clamp with the partial loop grasping head 305 having a comparatively smaller lateral access aperture 306 and shorter perimeter 307 than the partial loop grasping head depicted in either FIG. 3A or FIG. 3B.

In some examples, as seen in FIG. 3C, the lateral access aperture 306 on the partial loop grasping head portion 305 is positioned on the contralateral aspect of the laterally curved distal shaft portions 310. This lateral access aperture 306 facilitates unimpeded access to the clamped anatomical structure for suturing or stapling procedures.

In some examples, as seen in FIGS. 3A-3C, the partial loop grasping head portion 305 opens laterally, either to the left or right side relative to the handle.

In some examples, as seen in FIGS. 3A-3C, the partial loop grasping head portion 305 is situated at an angle between 0 and 90 degrees relative to the longitudinal axis of the laterally curved distal shaft portion 310.

In some examples, as seen in FIGS. 3A-3C, the partial loop grasping head portion 305 provides a lateral access aperture 306 which can be utilized for the insertion of objects or instruments into the perimeter 307 of the clamped tissue area. The partial loop grasping head portion 305 is further customizable by adjustments to perimeter 307 and lateral access aperture 306 lengths.

For example, users requiring clamping of a larger surface area will desire a longer length perimeter 307 for the partial loop grasping head structure 305.

According to certain examples, a wider lateral access aperture 306 will provide broader spatial access for inserted instruments of varying sizes. Both the perimeter 307 and lateral access aperture 306 may be individually customizable to benefit a wide range of surgical applications and individual user preferences.

According to certain examples, as seen in FIGS. 3A-3C, the partial loop grasping head portion 305 of the invention may comprise: a round-, ovoid-, or oval-shaped perimeter 307 between 5 mm and 30 mm, having a radius of curvature between 2 mm and 15 mm; and a lateral access aperture 306 configured to provide access for suturing or stapling tissue, the aperture having a width between 2 mm and 15 mm.

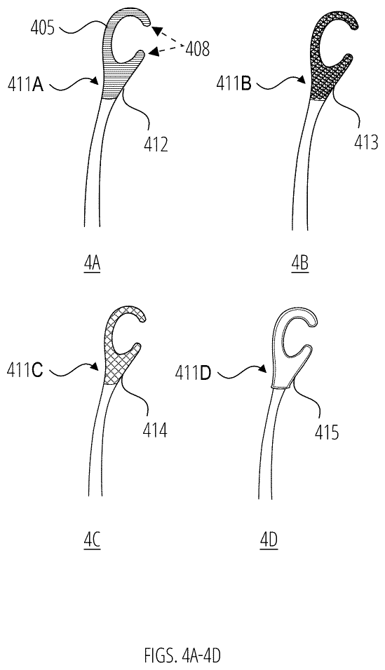

FIGS. 4A-4D are diagrams depicting different textured configurations of the inner surface of the partial loop grasping head of the surgical clamp, according to certain examples.

FIG. 4A shows the inner surface 411A of the partial loop grasping head 405 textured with serrations 412. In some examples, these serrations 412 have a depth between 0.01 mm and 1 mm and enhance friction on clamped tissues.

FIG. 4B shows the inner surface 411B of the partial loop grasping head 405 textured with toothing 413. This toothed configuration provides improved grip on tissues while maintaining atraumatic edges 408 for delicate grasping and manipulation of friable tissues.

FIG. 4C shows the inner surface 411C of the partial loop grasping head 405 textured with crosshatching 414. In some examples, the crosshatched pattern has a depth between 0.01 mm and 1 mm and offers enhanced friction and tissue control during surgical procedures.

FIG. 4D shows the inner surface 411D of the partial loop grasping head 405 textured with rubber 415. In some examples, this rubberized surface has a thickness between 0.1 mm and 2 mm, offering improved grip and reduced trauma to the clamped tissue.

In some examples, the partial loop grasping head portions 405 are outfitted with textured inner surfaces (411A, 411B, 411C, 411D) and one or more atraumatic edges 408 for delicate grasping and manipulation of friable tissues. The one or more atraumatic edges 408 serve to blunt the frictional impact of the tool on adjacent tissues during clamping.

In certain examples, the inner surfaces of partial loop grasping head portions 405 can be configured with various textures as depicted in FIGS. 4A-4D. These textured inner surfaces include serrations 412, toothing 413, crosshatching 414, or rubber 415 to modulate friction on delicate tissue and augment the surgeon's ability to manipulate clamped tissues with minimal physical exertion. The shape, texture, and edges of the first and second partial loop grasping head portions 405 provide control during tissue manipulation while minimizing tissue damage.

In some examples, as seen in FIGS. 4A-4C, the inner surfaces (411A, 411B, 411C) of the partial loop grasping head portions are textured to enhance friction on clamped tissues and augment the surgeon's ability to manipulate clamped tissues with minimal physical exertion. The inner clamping surfaces of the first and second partial loop grasping head portions comprise different configurations to suit various surgical needs.

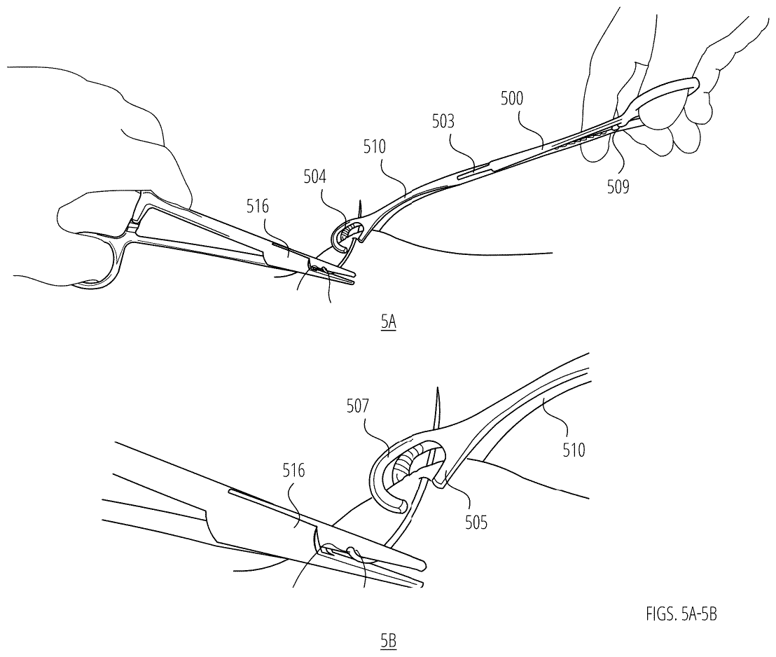

FIGS. 5A-5B provide diagrams depicting a surgical clamp, according to certain examples.

The tool comprises first and second shafts 500 joined at a hinge 503, which may be locked together via a ratchet 509. At the distal end of the laterally curved distal shaft portions 510, first and second jaw portions 504 further comprise first and second partial loop grasping head portions 505 that provide utility during the insertion and stabilization of surgical instruments 516 or endoscopy equipment into tissue structures. The partial loop grasping head portions 505 define a perimeter 507 that creates a semi-enclosed space for tissue manipulation.

In some examples, the direction of the opening in the partial loop grasping head portions 505, the width of the perimeter 507, and the inclusion of laterally curved distal shaft portions 510 are customized according to surgical specialty to optimize access and manipulation in confined surgical spaces.

As illustrated in FIGS. 5A and 5B, the surgical clamp provides stabilization of local anatomical structures while preserving spatial accessibility to the tissue site for placement of sutures or staples. This design addresses limitations of conventional clamping devices that typically provide a full circular grip around the intended structure, which can be obstructive in confined spaces or when dealing with delicate tissues.

In some examples, clamping with the first and second partial loop grasping head portions 505 can be achieved simultaneously with co-insertion of another instrument 516 into the clamped tissue area, such as surgical needles carrying medical-grade sutures. This simultaneous clamping and access capability enhances procedural efficiency and precision.

In some examples, the first and second partial loop grasping head portions 505 can provide utility in aiding with the insertion and stabilization of endoscopy equipment into luminal structures, offering improved control during minimally invasive procedures.

According to certain examples, the invention can be used in a method for clamping an anatomical structure during a surgical procedure, the method comprising:

Providing a surgical clamp with first and second partial loop grasping head portions 505 oriented in a non-planar configuration relative to first and second handle portions, the first and second partial loop grasping head portions having a perimeter 507 between 5 mm and 30 mm and a lateral access aperture width between 2 mm and 15 mm;

Positioning the first and second partial loop grasping head portions 505 around the anatomical structure to provide lateral and proximal clamping; and

Utilizing the lateral access aperture of the first and second partial loop grasping head portions 505 to access and ligate the anatomical structure with a suture or staple 516 having a diameter or width between 0.1 mm and 2 mm.

The method may further include applying directional clamping pressure to the anatomical structure using the first and second partial loop grasping head portions 505, wherein the clamping pressure is between 0.1 N/mm² and 10 N/mm².

The first and second partial loop grasping head portions 505 allow surgeons to apply precise clamping pressure from a specific direction, which is particularly useful in procedures where a full circular grip could be obstructive or less effective, thereby enhancing surgical precision and reducing procedural complications.

What is claimed is:

1. A tissue-grasping medical clamping tool, the tool comprising:
elongated first and second shafts, respectively including corresponding proximal first and second handle portions and corresponding distal grasping first and second jaw portions that meet to define a grasping plane, a medial portion of the first and second shafts including a hinge arranged for interlocking of the first and second shafts; and
first and second jaw portions respectively including corresponding first and second partial loop grasping head portions aligned with the grasping plane, each of the first and second partial loop grasping head portions comprising a lateral access aperture oriented in the same lateral direction to permit an instrument to be laterally inserted into a perimeter defined by the first and second partial loop grasping head portions.

2. The tool of claim 1, wherein the first and second partial loop grasping head portions comprise a textured inner surface that comprise at least one of serration, toothing, or crosshatching.

3. The tool of claim 2, wherein a depth of the serration, the toothing or the crosshatching is between 0.01 mm and 1 mm.

4. The tool of claim 1, wherein the first and second partial loop grasping head portions comprise a smooth inner surface.

5. The tool of claim 1, wherein the first and second partial loop grasping head portions comprise a rubberized surface.

6. The tool of claim 5, wherein the rubberized surface of the first and second partial loop grasping head portions is between 0.1 mm and 2 mm in thickness.

7. The tool of claim 1, wherein the first and second partial loop grasping head portions comprise one or more atraumatic edges.

8. The tool of claim 1, wherein the first and second handle portions comprise a ratchet for incremental tightening between the first and second partial loop grasping head portions.

9. The tool of claim 8, wherein the ratchet exerts a directional clamping pressure between 0.1 N/mm² and 10 N/mm².

10. The tool of claim 1, wherein the first and second shafts comprise at least one of stainless steel, titanium, carbon steel, plastic, ceramic, silicone, rubber or glass.

11. The tool of claim 1, wherein the lateral access aperture provides lateral access to the left or right side of the first and second handle portions.

12. The tool of claim 1, wherein the lateral access aperture provides lateral access at an angle between 0 degrees and 90 degrees relative to the longitudinal axis of the first and second handle portions.

13. The tool of claim 1, wherein the lateral access aperture measures between 2 mm and 15 mm.

14. The tool of claim 13, wherein the lateral access aperture permits lateral insertion of an instrument having a diameter or width between 0.1 mm and 2 mm.

15. The tool of claim 1, wherein the perimeter of the first and second partial loop grasping head portions measures between 5 mm and 30 mm.

16. The tool of claim 1, wherein the first and second shafts comprise laterally curved distal shaft portions.

17. The tool of claim 16, wherein the laterally curved distal shaft portions form an angle between 0 degrees and 45 degrees relative to the longitudinal axis of the first and second handle portions.

18. The tool of claim 1, wherein the hinge comprises one or more of a pivot, box or spring mechanism.

19. The tool of claim 1, wherein the grasping plane is non-planar to the plane of the first and second handle portions.
